(19) United States
(12) Patent Application Publication
(10) Pub. No.: US 2026/0252045 A1
(43) Pub. Date: Aug. 27, 2026

(54) INTELLIGENT AGENT FOR GUIDING DEVELOPMENT OF COMPOSITE PRODUCTS

(71) Applicant: Osmo Labs, PBC, New York, NY (US)

(72) Inventors: Alexander Wiltschko, Somerville, MA (US); Roland Jasper Snoek, Cambridge, MA (US); Ben Michael Edwin Adlam, Boston, MA (US); Benjamin Manuel Sanchez-Lengeling, Somerville, MA (US)

(21) Appl. No.: 18/994,144

(22) PCT Filed: Jul. 14, 2023
(86) PCT No.: PCT/US23/27801

§ 371 Date: Jan. 14, 2025

Related U.S. Application Data
(60) Provisional application No. 63/389,517, filed on Jul. 15, 2022.

(51) Int. Cl. G05B 13/04

(52) U.S. Cl. CPC G05B 13/041

(57) ABSTRACT

The present disclosure is directed to systems and methods enabling an intelligent agent to optimize a black-box objective function over a sufficiently large search space. More particularly, the intelligent agent may be employed to provide guidance in the design, development, and/or formulation (or a reformulation) for novel (or pre-existing) products. The intelligent agent employs machine learning and statistical techniques to provide the guidance. A user and the intelligent agent may communicate through a “smart spreadsheet” enabled by the embodiments.

### PRIORITY APPLICATION

The present application claims the benefit of U.S. Provisional Patent Application No. 63/389,517, filed on Jul. 15, 2022, the disclosure of which is hereby incorporated by reference herein in its entirety.

### FIELD

The present disclosure relates generally to the development of composite products. More particularly, the present disclosure relates to providing automated and intelligent guidance to assist in the development of products manufactured by combining a plurality of ingredients, for which the resulting properties of are not a priori known.

### BACKGROUND

Many products such as food items, beverages, cosmetics, fragrances, pharmaceuticals, and the like are manufactured by combining a subset of “ingredients” from a set of possible ingredients in various relative ratios. The combination of the ingredients in various ratios often results in synergistic effects, such that properties relating to human perception (e.g., taste, flavor, smell, color, texture, viscosity, and the like) may not be readily inferable from a “recipe” that describes the ingredients and the relative ratios. As such, artisans, craftsmen, and technicians who are tasked with developing novel products often undertake an arduous trial and error-based effort to painstakingly create new recipes that satisfy emerging fashions, tastes, and preferences in intended consumers, while simultaneously meeting marketplace constraints, such as production cost, dietary restrictions, and governmental regulations.

Via such trial-and-error efforts, some industries have generated “rules of thumb,” and other heuristic mechanisms that provide some guidance when designing novel products. However, the applicability of such heuristics may not readily scale as consumers continue to seek novelty in products, with swift volatility in marketplace constraints (e.g., costs), and as additional ingredients are continually added to the set of possible ingredients.

### SUMMARY

Aspects and advantages of embodiments of the present disclosure will be set forth in part in the following description, or can be learned from the description, or can be learned through practice of the embodiments.

One example aspect of the present disclosure is directed to a computer-implemented method for mixture property prediction. The method can include receiving an indication of a set of potential formulations for the target product. An indication of target values for a set of relevant properties for the target product may also be received. An objective function over a first domain may be generated. The objective function may be associated with the set of relevant properties. The target values for the set of relevant properties may optimize the objective function over the first domain. A set of estimating functions may be determined. The set of estimating functions may enable estimating (e.g., a variance of) the objective function over a second domain. The second domain may be associated with a set of potential functions. Determining the set of estimating functions may be based on first property data encoding values for the set of relevant properties. The values for the set of relevant properties encoded by the first property data may be associated with a first subset of the set of potential formulations. The set of estimating functions may be iteratively updated based on additional property data. The additional property data may encode additional values for the set of relevant properties. The additional values for the set of relevant properties encoded by the additional property day may be associated with additional subsets of the potential formulations. A target formulation may be selected from the set of potential formulations. Based on the updated set of estimating functions, the target formulation may substantially optimize the objective function over the second domain.

Another example aspect of the present disclosure is directed to another a computer-implemented method for mixture property prediction. The other method can include receiving, via a user interface (UI) provided by a computing system, first product data for a property of the target product. The first property data may correspond to a first potential formulation of a set of potential formulations for the target product. A second potential formulation may be selected from the set of potential formulations. Selecting the target formulation may be based on processing, at the computing system, a first evaluation of an objective function. The objective function may be defined over the set of potential formulations. The objective function may be based on a target value for the property. The first evaluation of the objective function may be based on the first property data and may be associated with the first potential formulation. Second property for the property of the target product may be received via the UI. The second property data may correspond to the second potential formulation. A second evaluation of an objective function may be processed at the computing system. The second evaluation of the objective function may be based on the second property data and may be associated with the second potential formulation. A target formulation may be from the set of potential formulations. Selecting the target formulation may be based on processing the second evaluation of the objective function. The target formulation may be provided via the UI.

Any two or more of the features described in this specification, including in this summary section, may be combined to form implementations of the disclosure, whether specifically expressly described as a separate combination in this specification or not.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 depicts a block diagram of an example computing system that enables the formulation of a target product according to example embodiments of the present disclosure;

FIG. 2 depicts a block diagram of an example intelligent agent engine that performs according to example embodiments of the present disclosure;

FIG. 3A depicts a flow chart diagram of an example method for providing guidance in the formulation of a target product according to example embodiments of the present disclosure; and

FIG. 3B depicts a flow chart diagram of an example method for iteratively optimizing an objective function according to example embodiments of the present disclosure.

Reference numerals that are repeated across plural figures are intended to identify the same features in various implementations.

### DETAILED DESCRIPTION

### Overview

Generally, the present disclosure is directed to systems and methods enabling an intelligent agent to (at least approximately) optimize (e.g., minimize or maximize) a black-box objective function over a sufficiently large search space. More particularly, the intelligent agent may be employed to provide guidance in the design, development, and/or formulation (or a reformulation) for novel (or pre-existing) products. The product to be formulated or reformulated may be referred to as the “target product”. The target product may be a composite product. Non-limiting examples of types of composite products include foodstuffs, food items, beverages, cosmetics, fragrances, pharmaceuticals, fuel, and the like. Other non-limiting examples of types of composite products include consumer products such as pesticides, insect repellents, and the like.

As discussed below, the embodiments may model the formulation of a target product as a search problem over a high dimensional (and sufficiently large) search space. Such a search space may be a vector space, where a set of potential formulation vectors encoding a set of potential formulations for the target product spans the vector space. The “search criteria” may be defined, via an objective function, to locate potential formulations (within the search space) that at least approximately optimize target values for a set of relevant properties for the target product. The search may be an interactive and iterative search over the space. The interactivity of the search may be between the intelligent agent and a user (e.g., a party that is interested in the formulation of the target product). For each iteration of the search, the intelligent agent may provide guidance by intelligently recommending sets of points in the search space for a user to “experimentally sample.” The user may experimentally sample the recommended points and provide the intelligent agent with the experimental results. Based on the experimental results, the intelligent agent may recommend a next set of sampling points. This process may be iterated until the search converges to locate a point in the space that at least approximately satisfies the search criteria (e.g., a point that at least approximately optimizes the objective function).

More particularly, the guidance provided by the intelligent agent may include iterative recommendations of one or more points to empirically (or experimentally) sample in the space of potential formulations. Sampling a point in the space of potential formulations may include manufacturing or preparing a prototype version of the target corresponding to the sampling point (e.g., manufacturing a prototype product based on the potential formulation corresponding to the sampling point). Once the prototype product has been manufactured, its unpredictable (and relevant) properties may be empirically (or experimentally) measured or observed.

The process of manufacturing a prototype product, via a potential formulation, and empirically observing values for its corresponding unpredictable (and relevant) properties may be referred to as an “experiment” on the potential formulation or an “experimental” observation of the corresponding potential formulation. The intelligent agent may empirically evaluate the objective function at each of the sampling points based on the empirically measured unpredictable properties, as well as any predictable properties of interest, (e.g., relevant properties). The space of potential formulations (e.g., the search space) may be referred to as simply the “formulation space,” or “the space of formulations.” Vectors in the formulation space may be referred to as “potential formulation vectors,” or simply “formulation vectors.”

As with other search problems, an objective function is employed to quantify how well a particular point in the search space satisfies the objective of a search criteria. For the embodiments, the search criteria may include matching (as close as possible) values for relevant properties associated res from a potential formulation with target values for the set of relevant properties for the target product. The search over the search space may be constrained by a set of design constraints. For instance, the sampling of the space of potential formulations and optimization of the objective function may not consider one or more sub-regions of the space that violate one or more design constraints of the set of design constraints.

A scalar objective function may be defined over the space of potential formulations. When constructing such a space of potential formulations, the intelligent agent may automatically remove any regions of the space that correspond to a potential formulation that violates one or more design constraints. Such a vetoing of such regions ensures that the intelligent agent does not recommend sampling points that correspond to potential formulations that would violate the design constraints. The objective function may be a function of the potential formulations for the target product. The objective function may indicate a concordance between values observed for a set of relevant properties associated with the potential formulation and target values (e.g., intended or desired values) for each relevant property of the set of relevant properties for the target product. The objective function may be defined (e.g., in view of the target values for the set of relevant properties), such that a point in the formulation space that at least an approximately optimizes (e.g., a maximization or minimization) the objective function corresponds to a potential formulation that results in a target product with properties that are in at least partial concordance with the target values for the set of relevant properties.

That is, the “objective” of the objective function is to select an at least somewhat “optimized” formulation from the set of potential formulations. The selected formulation enables the production of a target product with properties that are in concordance with target values for the relevant properties for the target product. The at least somewhat optimized formulation may be referred to as a “target” formulation. The embodiments may model the formulation of a target product as a search for a target formulation within a large search space comprising the set of potential formulations.

A set of relevant properties may be defined by the user. Each property in the set of relevant properties may be a property that is to be controlled (e.g., selected for or against) in the formulation of the target product. The set of relevant properties (and associated values for each relevant property) may define a “relevant property space.” Each dimension in the relevant property space may correspond to a relevant property in the set of relevant properties. The basis vectors (and associated metric) associated with dimensions in the space may correspond to a quantification of the corresponding relevant property. The relevant property space may be simply referred to as the “property space.” Thus, a quantification of set of relevant properties corresponding to a potential formulation may be encoded in a property vector embedded in the property space. As discussed below, user-provided target values for the set of relevant properties may be encoded in a target property vector (or set of target property vectors) in the property space. Thus, the search problem may be defined as a search for a potential formulation that is associated with a vector embedding in the property space that is “similar” to the target property vector.

In at least one embodiment, the intelligent agent may encode the target values for the set of relevant properties in a “target property vector” embedded in the property space. Each formulation vector in the formulation space may correspond to a property vector in the property space, via the values of the relevant properties that the potential formulation would generate. More particularly, potential formulations may generate a prototype product with values for the relevant properties that correspond to points in the property space. Thus, a mapping between points in the formulation space and points in the property space may exist. Note that this mapping may not be a one-to-one mapping, in that there may be one or more points in the property space that are not achievable via a potential formulation in the formulation space. Furthermore, there may be more than one potential formulation that corresponds to the same point in the property space, e.g., multiple potential formulations result in products that have equivalent (or at least similar) properties.

Because the objective function is a function of both points in the formulation the formulation space and the target values for the set of relevant properties, this mapping may be useful to evaluate the objective function. However, because one or more relevant properties may be unpredictable, the mapping from formulation space to property space may not be feasible via predictive means. Empirical or experimental means may be required to generate the mapping from the formulation space to the property space for a target product. Thus, empirical means may be required for evaluation of the objective function.

As noted above, the objective function may be based on target values for a set of relevant properties, e.g., the vector embedding(s) of target values for the set of relevant properties. As also noted above, at least some of the relevant properties may be unpredictable. Due to the unpredictability of at least some of the target/relevant properties, the objective function may be a black-box function. That is, generating a mapping of a formulation to values for its corresponding relevant properties based on first principles, modeling, or other predictive means may be unfeasible. Thus, the objective function may be a “black-box function.” The evaluation of such a black-box objective function at a generalized point in the formulation space may not be feasible from a predictive analysis.

Rather, evaluating the objective function at a point in the formulation space may require empirical (or experimental) mechanisms. As such, the optimization of the objective function may require empirical sampling of the objective function at a finite set of sampling points to generate an estimate of the objective function over the formulation space. A target formulation (e.g., a potential formulation that at least approximately optimizes the objective function) may be selected via the empirical evaluations at the sampled points and/or the estimate of the objective function over the formulation space.

Such empirical evaluations of the objective function may be expensive in both time and financial resources. Accordingly, the intelligent agent provides intelligent guidance (or suggestions) by iteratively recommending one or more sets of sampling points to sample in the formulation space. When the sampling points are intelligently selected by the intelligent agent, performing iterative empirical evaluations of the objective function at the recommended sampling points enables efficient convergence of estimating the objective function. Once its estimate has converged, the objective function may be at least approximately optimized, enabling a selection of a target formulation that is in concordance with the target values for the set of relevant properties. Performing the empirical observations at the sampling points recommended by the intelligent agent enables significantly greater efficiency in the convergence of the estimation of the objective function, as well as the optimization of the objective function, as compared to conventional trial-and-error or heuristic-based methods. That is, the embodiments enable the formulation of a target product in a significantly more efficient manner than conventional methods.

The intelligent agent may employ machine learning and/or statistical-based methods to determine recommended sets of points to sample in the space of formulations. In at least one embodiment, the intelligent agent may employ one or more black-box optimization techniques to generate the recommended one or more sets of sampling points. Such employable black-box optimization techniques include, but are not limited to, Bayesian optimization and/or Gaussian process regression techniques.

The formulation process (e.g., the optimization of the black-box objective function) may be an iterative process. For instance, a party interested in the formulation of a target product may sample an initial set of sampling points by empirically observing the values for the relevant properties of prototype products associated with potential formulations corresponding to the set of initial sampling points. In such embodiments, the party may provide the initial set of sampling points and the empirically observed values of the relevant properties at each of the initial sampling points. The intelligent agent may evaluate the objective function at these initial sampling points based on being provided the empirically observed values for the properties. Based on the evaluation of the objective function at the initial set of sampling points, the intelligent agent may employ a black-box function optimization technique and the set of initial sampling points to generate a statistical bounding (e.g., a variance) on a set of potential functions that at least approximates the objective function to a particular confidence level. The set of potential functions may be considered as a statistical estimate of the objective function. The statistical bounding of the set of potential functions is employed to select (and recommend) a next set of sampling points, which will improve the estimation of the objective function. The interested party may perform empirical evaluations of the relevant properties at the recommended set of sampling points. The party may then provide the empirical observations of the values of the relevant properties at the recommended next set of sampling points to the intelligent agent. This process may iteratively continue until the estimation of the objective function converges to a convergence level that admits to at least a partial optimization of the objective function. Such an optimization of the objective function enables the agent to select a target formulation from the set of potential formulations.

The intelligent agent and the interested party (e.g., a user) may iteratively communicate during the formulation process via a user interface (UI) provided by the intelligent agent. In non-limiting embodiments, the UI interface may be a graphical user interface (GUI). In at least one embodiment, the GUI may be a “smart spreadsheet” that includes an interactive table. The table may include a set of rows and a set of columns that form a set of cells. Each cell may be uniquely indexed by a corresponding row and column. Each row may correspond to a potential sampling point in the formulation space. At least a portion of the columns may be employed to storing values that encode (recommended and/or sampled) potential formulations. Another portion of the columns may correspond storing values that encode property vectors representing the set of relevant properties for a potential formulation that has been subject to an “experimental observation” of sampling. For example, a user that has made an experimental observation of a prototype product (manufactured via a corresponding potential formulation) may enter the experimentally observed values of the relevant properties into the cells corresponding to the row and columns for that experiment. In this way, the user may provide “experimental results” associated with sampling to the intelligent agent.

Another portion of the columns may be employed to store values encoding a potential formulation, e.g., a formulation vector. The intelligent agent may recommend sampling points to the user by “filling in” the “formulation encoding cells” of a row corresponding to a recommending sampling point in the formulation space, with the formulation. In at least one embodiment, the intelligent agent may highlight rows corresponding to recommended sampling points. At least one column may be employed to store a value that indicates the empirical evaluation of the objective function. In such embodiments, the intelligent agent may automatically provide the value of the evaluated objective function in the cell corresponding to the sampling point and the corresponding column.

The intelligent agent may provide recommended formulations to the user via the GUI. Likewise, the user may provide potential formulations to the intelligent agent via the GUI. The user may provide the set of potential formulations, the set of relevant properties, target values for the set of relevant properties, and the set of design constraints to the intelligent agent via the GUI. The user may also provide the intelligent agent with experimental results of sampling (e.g., empirical observations of the quantification of relevant properties) via the GUI. The intelligent agent may provide the user with empirical evaluations of the objective function, as well as recommended sampling points via the GUI. In this way, the GUI may serve as an interactive interface between the intelligent agent and a party interested in formulating a target product.

As noted, at least due to the unpredictability of some properties, the formulation or reformulation of a novel or pre-existing product, via conventional methods, may involve a costly, laborious, and time-consuming trial-and-error-based effort. Even when conventional rules-of-thumb, heuristics, and other sorts of empirical (or experiential) knowledge informs the formulation process, the development (or redevelopment) of products that are favorable to consumers' expectations and tastes, via conventional methods, may be cumbersome, expensive, and largely non-successful. The embodiments overcome such limitations associated with conventional methods by providing an enhanced machine learning and statistical-based approach to such formulation or re-formulation efforts. In addition to overcoming such conventional limitations, the embodiments provide a number of technical effects and technical benefits, as discussed throughout.

For purposes of this disclosure, a composite product may be any tangible or virtualized object comprised of a plurality of other object types, items, elements, components, compounds, alloys, chemicals, molecules, and/or atoms. That is, a composite product is any (tangible or virtual) object that is comprised of two or more sub-objects or sub-components. Composite products may include consumable items such as, but not limited to, foodstuffs, food items, beverages, cosmetics, fragrances, pharmaceuticals, fuel, and the like. Other non-limiting examples of composite products include consumer products such as pesticides, insect repellents, and the like. Unless stated to the contrary, the terms “complex product,” “product,” “composite,” and “compound” may be employed interchangeably to refer to a composite product.

As used herein, the term “ingredient” of a product may refer to a particular sub-object or sub-component that is included in a product. An ingredient of a product may include any object, item, element, component, compound, alloy, chemical, molecule, and/or atom included in the product. For the non-limiting example of a soft drink-based product, sugar is one ingredient of the soft drink. As used herein, the terms “set of ingredients” or “list of ingredients” for a product may be employed to refer to a set of information that indicates a set of ingredients for the product. In the soft drink example, the set of ingredients for the soft drink may include but is not necessarily limited to: {carbonated water, sugar, caramel color, phosphoric acid, caffeine, flavor_1, flavor_2}.

As used herein, the term “formulation” for a product may be employed to refer to a set of information that indicates a set of ingredients for a product, as well as the absolute and/or relative amounts of each ingredient of the set of ingredients that are needed for the manufacture, fabrication, and/or preparation for the product. A formulation for a product may encode the molecular or chemical composition (e.g., a chemical formula) of the product. In the soft drink example, a formulation for the soft drink may include information such as: {(carbonated water, amt₁), (sugar, amt₂), (caramel color, amt₃), (phosphoric acid, amt₄), (caffeine, amt₅), (flavor_1, amt₆), (flavor_2, amt₇)}, where amtx represents an absolute or relative amount (or ratio) of the corresponding ingredient needed for the manufacture of the soft drink. In at least one embodiment, a product's formulation may encode or include information indicating instructions for combining (or compiling) the ingredients in a manner that at least partially enables the manufacture or preparation of the product. That is, a formulation of a product may enable the manufacture or preparation of the product. Thus, a product's formulation may encode at least somewhat enabling instructions for its manufacture or preparation. The terms “recipe,” “bill of materials,” “BOM,” “blueprint,” “source code,” “design,” or “genotype” for a product may be used interchangeably to refer to the product's formulation. As discussed throughout, a formulation for a product may be encodable in a data structure. In at least one embodiment, a formulation may be encoded in a multi-dimensional data object or structure, such as but not limited to a vector, a 1D array, or an n-tuple. In some embodiments, a formulation may be encodable in higher-dimensional data objects or structures, such as but not limited to a 2-rank tensor (e.g., a matrix), higher rank tensor and/or array objects.

The term “property” of a product may include any observable, perceivable, measurable, and/or quantifiable aspect, characteristic, or feature of the product. In a loose analogy, if a formulation (or recipe) for a product is analogized as a “genotype” for the preparation and/or manufacture of the product, a property of the product may be analogized to an “phenotype” of the manufactured/prepared product. Accordingly, in a similar manner to how a product's formulation may be referred to as the product's “genotype,” a property of the product may be referred to as a “phenotype” of the product. In another loose analogy, if a product is a software application and its formulation is its source code, a property of the product may be a feature (or a bug) in the compiled application. Properties of a product include but are not limited to human perceptions associated with consumption of the product: e.g., taste, flavor, smell, color, texture, viscosity, and the like. Other example properties of a product may include an effectiveness of the product's intended purpose, (e.g., does an insect repellent repel insects), a (relative or absolute) cost of the product's formulation, or whether the formulation is compliant with one or more regulations, dietary/nutritional restrictions, and other such constraints. Still other example properties of a product include nutritional information (e.g., energetic, macronutrient, and/or micronutrient quantities), mass, volume, and the like.

Some (but not necessarily all) properties may be quantifiable to various levels of resolution. For instance, some properties may be quantified via a binary classification (e.g., does the product's formulation include (or not include) a particular ingredient, is the formulation compliant with a particular regulation, or does the insect repellent repel insects). Some example properties may be quantified with an unbounded (or bounded) and/or continuous (or discretized) scalar value (e.g., a formulation's cost, the cardinality of the formulation's set of ingredients, or how well the insect repellent repels insects). Still other example properties may be quantified with higher dimensional objects, e.g., (1D or multi-D) arrays, n-tuples, vectors, matrices, higher-order tensor objects, and the like. For instance, a taste of a product may be quantifiable via a vector, 1D array, or n-tuple, where the different sense (or domains) of tastes (e.g., sweet, sour, salty, bitter, and umami) correspond to separate dimensions or components of the vector, array, or n-tuple. Note that an encoding of a set of relevant properties for a product may not encode all properties of the product. For example, the encoding may encode only a subset of the properties that are relevant (e.g., target values for a set of relevant properties).

Some properties may submit to (at least somewhat) objective quantification, e.g., the formulation's costs, nutritional information, a pungency (e.g., spiciness or “heat”) of the product (e.g., the Scoville scale) or does the formulation include (or not include) a particular ingredient. A quantification of other properties may be more subjective. For instance, a taste, flavor, or fragrance of the product may be subjective and vary from individual-to-individual opining on the property. The “nose” or “bouquet” properties associated with wine is an example of a product property that tends to be more subjective, and less objective, than other properties.

Some product properties may be (at least somewhat) predictable, inferable, or otherwise determinable based at least partially on knowledge of the product's formulation and/or other properties. That is, some properties may be at least somewhat predictable from its formulation prior to manufacturing or preparing at least a prototype version of the product in view of theories, first principles, first order (or linear) effects, prior knowledge (e.g., experience), heuristics (e.g., rules-of-thumb), and/or models that are readily applicable to the product formulation. For instance, the cost, nutritional information, and mass/volume of a soft drink product may be predictable or inferable from the product's formulation. Other properties may be less predictable or inferable via the product's formulation. Due to unknown interactions, synergistic effects, non-linear relationships, and other unpredictable and/or uncontrollable interactions between ingredients, some product properties may be at least somewhat unpredictable prior to a manufacture or preparation of at least a protype of the product. The unpredictability of at least some properties may be at least partially due to the property's inherent subjectivity, e.g., a variance or variation in how a population of tasters may subjectively rate a taste of a product. Properties associated with large variations (or variances) may be classified as unpredictable properties. Some properties may be at least somewhat unpredictable because the property is at least partially due to unmodeled interactions between ingredients, second (or higher)-order effects between the ingredients, or other such unknown mechanisms. Some unpredictable or non-inferable properties may be “emergent” in that the “emergent property” does not present itself until the ingredients are brought together or combined as encoded in the product's formulation.

The terms “empirical,” “unpredictable,” “non-inferable” or “emergent” may be employed interchangeably to refer to a property that is not readily predictable or inferable via a product's formulation, theories, first principles, prior knowledge (e.g., experience), heuristics (e.g., rules-of-thumb), and/or models that are readily applicable to a potential product formulation. Properties that are readily predictable or inferable via first principles, theoretical considerations, experience, heuristics, models, and the like may simply be as a “predictable” or “inferable” property. Note that the characterization of a particular property as a predictable property or an unpredictable property may be itself an unpredictable property of the product's property. For example, due to unknown or unmodeled interactions between ingredients, prior to manufacturing or preparing at least a prototype of the product, whether a particular property is unpredictable or predictable may be itself unpredictable. The predictability of whether one or more properties of a product is unpredictable may be said to be a “meta-property” (or just simply a property) of the property. Properties with unpredictable meta-properties may be categorized as unpredictable.

### Example Devices and Systems

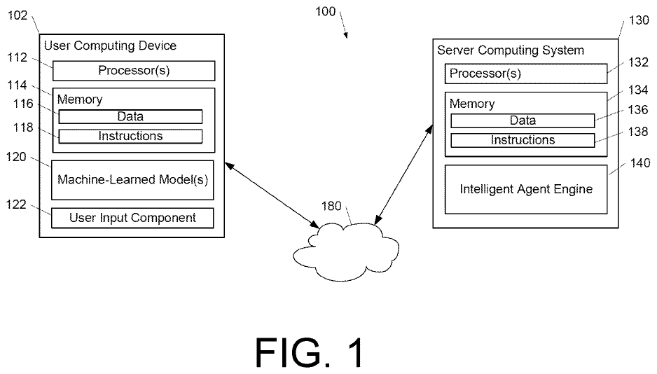

FIG. 1 depicts a block diagram of an example computing system 100 that enables the formulation of a target product according to example embodiments of the present disclosure. The system 100 includes a user computing device 102 and a server computing system 130 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

More particularly, the machine-learned prediction model can be trained to intake molecule data and mixture data and output property predictions for the mixture the mixture data is descriptive of. In some implementations, the molecule data may be embedded with an embedding model before being processed by a prediction model.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

The server computing system 130 can include an intelligent agent engine 140 that is enabled to provide guidance in the development of complex products as discussed herein. That is, the intelligent agent engine 140 provides intelligent agent services that are enabled to carry out the various embodiments discussed herein. Via the communication network 180, a user of the user computer device may access the services of the intelligent agent. That is, the user computing device 102 may be a client of the intelligent agent engine 140 implemented by the server computing system 130. Other embodiments may not employ such a client/server architecture. For instance, the user computing device may implement the intelligent agent engine 140, such that the user of user computing device 102 may access the services of the intelligent agent.

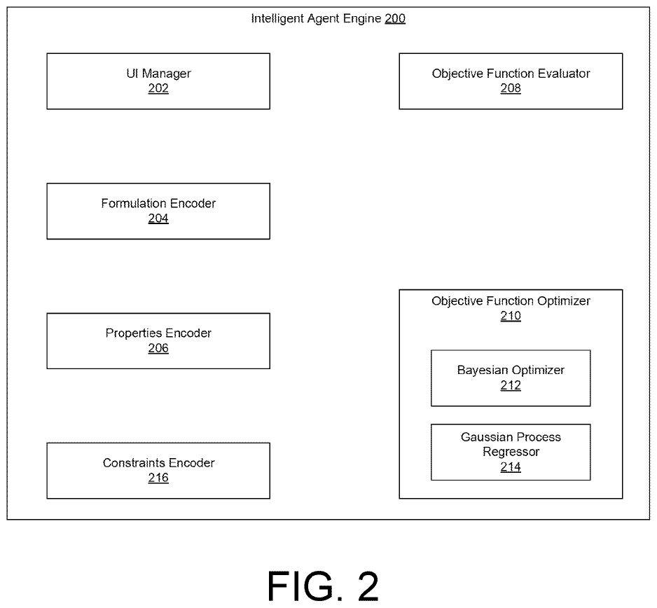

Various embodiments of an intelligent agent engine 140 are discussed in conjunction with at least FIG. 2. However, briefly here, the intelligent agent (e.g., implemented by the intelligent agent engine 140) may be enabled to optimize (e.g., minimize or maximize) a black-box objective function over a sufficiently large search space. More particularly, the intelligent agent may be employed (e.g., by a user of the user computing device 1023) to provide guidance in the design, development, and/or formulation (or a reformulation) for novel (or pre-existing) products. The product to be formulated or reformulated may be referred to as the “target product”. As discussed below, the embodiments may model the formulation of a target product as a search problem over a high dimensional (and sufficiently large) search space, e.g., a first vector space that is spanned by a set of potential formulation vectors encoding a set of potential formulations for the target product.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TICP/IP, HTTP, SMTP, FTP), encodings or formats (e.g., HTML, XML), and/or protection schemes (e.g., VPN, secure HTTP, SSL). FIG. 1 illustrates one example computing system that can be used to implement the present disclosure. Other computing systems can be used as well.

### Example Intelligent Agent Engines

FIG. 2 depicts a block diagram of an example intelligent agent engine 200 that performs according to example embodiments of the present disclosure. The intelligent agent engine 200 may be equivalent and/or similar to intelligent agent 140 of FIG. 1. Accordingly, the intelligent agent engine 200 may implement an intelligent agent. Whether implemented in user computing device 102 or server computing device 130 of FIG. 1, the intelligent agent may provide intelligent agent services to a user of user computing device 102. As shown illustrated in the non-limiting embodiment of FIG. 2, the intelligent agent engine 200 may include a user interface (UI) manager 202, a formulation encoder 203, and a properties encoder 206. Intelligent agent engine 200 may additionally include an objective function evaluator 208, an objective function optimizer 210, and a constraints encoder. The objective function optimizer 210 may include a Bayesian optimizer 212 and a Gaussian process regressor 214. Note that other embodiments of a intelligent agent engine 200 may include additional and/or alternative components, modules, and/or other structures.

The operations and functions of the non-liming example components of intelligent agent engine 200 are described below. However, briefly here, the UI manager 202 is generally responsible for providing one or more UIs to user computing device 102, as well as managing various interactions with the user computing device 102. The UI may include “smart spreadsheet” that enables interactions between the user and the intelligent agent. Formulation encoder 204 is generally responsible for encoding potential formulations in formulation vectors that correspond to points in the formulation space. Properties encoder 206 is generally responsible for encoding values (e.g., empirically observed and/or target values) for a set of relevant properties in a property vector. Constraints encoder 216 is generally responsible for encoding a set of design constraints. Objective function evaluator 208 is generally responsible for generating and evaluating an objective function, provided an encoding of a target values for the set of relevant properties, a formulation vector, and/or a empirically-observed property vector. Objective function optimizer 210 is generally responsible for optimizing the objective function. To perform its duties, objective function analyzer 210 may include a Bayesian optimizer 212 and a Gaussian process regressor 214. The Bayesian optimizer 212 is generally responsible for implementing a Bayesian optimization process that is employed to optimize the objective function. The Gaussian process regressor 214 is generally responsible for implementing a Gaussian process, which may be a sub-process of the Bayesian optimization process of the Bayesian optimizer 212.

The intelligent agent (implemented by the intelligent agent engine 200) is enabled to (at least approximately) optimize (e.g., minimize or maximize) a black-box objective function over a sufficiently large search space. More particularly, the intelligent agent may be employed to provide guidance in the design, development, and/or formulation (or a reformulation) for novel (or pre-existing) target products. The intelligent agent may model the formulation of a target product as a search problem over a high dimensional (and sufficiently large) search space, e.g., a first vector space that is spanned by a set of potential formulation vectors encoding a set of potential formulations for the target product. The guidance provided by the intelligent agent may include iterative recommendations of one or more points to empirically (or experimentally) sample in the space of potential formulations. Sampling a point in the space of potential formulations may include manufacturing or preparing a prototype version of the target corresponding to the sampling point, and empirically (or experimentally) measuring or observing the properties of the prototype product. As with other search problems, an objective function is employed to quantify how well a particular point in the search space satisfies the objective of the search. The search over the search space may be constrained by a set of design constraints. For instance, the sampling of the space of potential formulations and optimization of the objective function will not consider one or more sub-regions of the space that are indicated by the set of design constraints.

More particularly, the intelligent agent may be employed to provide guidance when attempting to introduce, increase, eliminate, or decrease one or more relevant properties of a novel or pre-existing target product. That is, the embodiments are employable when attempting to formulate (or reformulate) a target product, such that the formulation at least approximately maximally achieves target (or intended) values for a set of relevant properties for the target product. To such ends, a party (e.g., a user of the intelligent agent) interested in formulating (or reformulating) a target product (e.g., a developer, designer, preparer, manufacturer, distributor, publisher, customer, consumer, and the like) may define (via the UI) target values for a set of relevant properties for the target product. The set of relevant properties for a target product may include any of the properties to be introduced, increased, eliminated, or decreased in the formulation (or reformulation) of the target product. That is, the set of relevant properties may include any property to be constrained, controlled for, selected for, selected against, or otherwise targeted for in the target product. Note that the set of relevant properties may not be exhaustive of all the properties of the target product. Rather, the relevant properties of the set of relevant properties may be only those properties that are to be at least partially maximized, minimized, or selected for (or selected against) in the target product's formulation (or reformulation).

For one or more relevant properties of the set of relevant properties, a target value (e.g., a desired quantification) of the relevant property may be provided by the user. A target value of the relevant property (e.g., a target value) may include an intention to optimize (e.g., maximize or minimize) the relevant property. A relevant property (and/or its target value) may be an “intended” or “desired” property (and/or intended quantification of the relevant property) for the target product. The agent's guidance (e.g., recommending sampling points) will ensure that, upon manufacture or preparation, the arrived at formulation at least approximately maximally achieves the target values for the set of relevant properties. In a non-limiting example, a target product may be an insect formulation. In one non-limiting example, the target product may be an insect repellent. The set of relevant properties for the insect repellent may include: {repellency, cost}. The repellency may be a measurable scalar value that indicates how effectively the insect repellent repels insects. The cost may be a scalar value that indicates the cost of the set of ingredients included in the insect repellant's formulation. In some embodiments, the formulation of insect repellent may include attempting to at least approximately maximize the repellency of the insect repellent, while simultaneously constraining the cost to be less than a cost threshold.

Values (e.g., either empirically observed or provided target values) for the set of relevant properties may be encoded in a data structure or data object, e.g., a vector, 1D array, n-tuple, or the like. In such an encoding, each dimension (or component) of the vector (or array or n-tuple) corresponds to a relevant property and the value of the component may be set to a value (e.g., an empirically-observed or provided target value) of the relevant property. Other vector encoding schemas for a set of properties may be employed by the various embodiments. The vector encoding schema employed for values of a set of relevant properties may define a “property vector space” (or space of property vectors) for the target product and/or the set of relevant properties for the target product. In at least one embodiment, this vector space may be referred to as the property space for the target product. In non-limiting embodiments, each potential property may correspond to a dimension of the property vector space. The basis vector (and axis) for each dimension in the property vector space may correspond to the quantification of the property corresponding to the axis. Note that each point in the property vector space may not be realizable via a potential formulation. In the above insect repellant example, a portion of the property vector space corresponding to high repellency and low cost may not be achievable via a realizable formulation for the insect repellant.

The quantification of at least some relevant properties may be predictable without the requirement of manufacturing or preparing at least a prototype instantiation of the product. Other relevant properties may be unpredictable. For such unpredictable relevant properties, at least a prototype instantiation of the product may be manufactured or prepared. Once manufactured or prepared via a potential formulation, the unpredictable property may be measured, observed, or otherwise quantified. In the above insect repellent example, the repellency of a potential formulation may be unpredictable, while the cost of the potential formulation is predictable via a combination of the cost of the individual ingredients of a potential formulation's set of ingredients. To measure the repellency associated with a potential formulation, a prototype of the insect repellency may be manufactured, and the associated repellency may be measured. In such a scenario, the quantification of the repellency property is empirically and/or experimentally evaluated or measured.

In addition to target values for a set of relevant properties, the interested party may define a set of design constraints for the target product. The constraints encoder 216 may encode the set of design constraints in in a data structure that the objective function evaluator 208 may evaluate, or otherwise parse. A design constraint in the set of design constraints may be a constraint on a target product's formulation and/or set of relevant properties. Non-limiting examples of such design constraints include that a formulation must (or must not) include a particular ingredient, nutritional information must be bounded by one or more thresholds, or the market cost of the set of ingredients must not exceed a cost threshold. In the above insect repellant example, a design constraint of the set of design constraints may include ensuring that the cost of the set of ingredients to be less than the cost threshold. The agent's guidance will ensure that the arrived at formulation (and values for its associated set of relevant properties) complies with the set of design constraints.

The embodiments may employ various combinations of machine learning and statistical-based techniques that enable the agent to provide guidance in the generation, determination, development, formulation, and/or reformulation of one or more possible formulations for a novel or pre-existing target product. Such machine learning and statistical-based techniques may include but are not necessarily limited to Bayesian optimization and Gaussian process regression techniques. In some embodiments, the agent employs Bayesian optimization and/or Gaussian process regressor techniques to recommend points in the potential formulation space to sample, via empirical evaluations of the set of relevant properties.

The formulation (or reformulation) of a novel (or pre-existing) target product may include target values for a set of relevant properties and/or a set of design constraints. Target values for the set of relevant properties are defined to “improve” the product” in one or more domains of product evaluations (e.g., price, consumer taste profiles, product effectiveness, governmental regulation, and the like). Such improvement-scenarios may include but are not limited to searching for an updated formulation to lower the cost of manufacture, to include one or more newly available ingredients in the formulation, or in response to changing consumer trends and/or regulatory policies. For example, if a cost of one or more ingredients in an existing formulation suddenly rises due to supply chain-related issues, the embodiments may be employed to search for an alternative ingredient that would provide for similar, or even enhanced, properties for the product.

Via the UI manager 202, a user of the user computing device 102 of FIG. 1 may define a set of potential formulations, target values for a set of relevant properties, a set of design constraints for the target product, and a set of design constraints, via interactions with the UI manager 202. In at least one embodiment, the user may define an objective function. In other embodiments, the intelligent agent may automatically define and/or generate the objective function based on the user-provided set of potential formulations, the set of relevant properties, the target values for the set of relevant properties, and/or the set of design constraints. The user may define these objects via any suitable encoding schema. In at least one embodiment, the user may define the set of potential formulations by providing each of the potential ingredients. In some embodiments, the user may further define the set of potential formulations by providing (absolute or relative) quantified bounds (e.g., upper and/or lower bounds) for each potential ingredient. As noted throughout, the formulation encoder 204 may be enabled to transform a potential formulation (and thus the the set of potential formulations) from a native data schema employed by the user to provide the set of potential formulations to a common encoding (e.g., a vector encoding) recognized by the other components of the intelligent agent engine 200. Likewise, properties encoder 206 and the constraints encoder are enabled to transform the user-supplied data into common data encodings recognized by the other components of the intelligent agent engine 200.

The information included in each of these user-based definitions is discussed in further detail below. However, briefly here, the set of potential formulations may include each possible formulation for the target product that may be considered by the intelligent agent. Via vector encodings, generated by the formulation encoder 204, a vector space of potential formulations may be defined from the definition of the set of potential formulations. The set of relevant properties may include a list of each property that is to be controlled and/or constrained during the formulation of the target product. That is, properties included in the set of relevant properties are properties to be optimized, selected for, or selected against by the intelligent agent. The target values for the set of relevant properties may be a quantification of an intended (or desired) value for each of the relevant properties in the formulation. In some embodiments, a target value for a particular relevant property may be an indication that a value associated with the particular relevant property is to be optimized (e.g., maximized or minimized) in the formulation of the target product.

Via vector encodings, generated by the properties encoder 206, a vector space of properties may be defined from the definition of the set of relevant properties. The objective function may be a scalar objective function defined over the vector space of potential formulations. The objective function may be a function of the potential formulations for the target product. The objective function may indicate a concordance between values for a set of relevant properties associated with the potential formulation and target values for a set of relevant properties. The objective function may be defined (e.g., in view target values for the set of relevant properties), such that at least an approximate optimization (e.g., a maximization or minimization) of the objective function may be indicative of a potential formulation that results in a target product with properties that are in at least partial concordance with target values for the set of relevant properties. That is, the “objective” of the objective function is to select for an at least somewhat “optimized” formulation from the set of potential formulations. As noted above, in some embodiments, the intelligent agent may automatically generate the objective function. In other embodiments, the user may at least partially define the objective function. For instance, a user may provide the distance or similarity metric that the objective function employs when attempting to control for the relevant properties. The set of design constraints may include one or more constraints on the potential formulations and properties associated with a product manufactured from the set of potential formulations.

Each potential formulation included in the set of potential formulations is a “possible,” “potential,” or “candidate” formulation for target product. Each potential formulation may include a set of ingredients and an absolute or relative quantification of the amount of each ingredient of the set of potential ingredients of the formulation. For some potential ingredients, the quantification may be binary (e.g., the potential ingredient is or is not included in the potential formulation). For other potential ingredients, the quantification may include a rational number indicating an absolute or relative amount of the ingredient included in the potential formulation. Inherent in the concept of the set of potential formulations is a set of potential ingredients for the target product. Each potential ingredient in the set of potential ingredients is an ingredient that is included in at least one potential formulation of the set of potential formulations. Note that to be included in the set of potential ingredients, a potential ingredient may be, but is not required to be, included in more than one potential formulation of the set of potential formulations. In various embodiments, the set of potential ingredients is a finite set, and thus there are a finite number of ingredients that are included in the set of potential ingredients.

As noted above, each potential ingredient in a potential formulation may include an absolute or relative quantification of the amount of the potential ingredient included in the potential formulation. The potential (or possible) ranges of such quantifications may be a finite range, e.g., a potential range of a quantification for any potential ingredient is bounded by a lower and an upper threshold. In some embodiments, the absolute (or relative) lower bound (or threshold) for a potential ingredient may be 0.0. For instance, when the quantification of the amount of a potential ingredient is set to 0.0 for a potential formulation, the potential ingredient is not included in the corresponding potential formulation. An upper bound (or threshold) for a potential ingredient may be dependent on the absolute or relative scale employed for the formulation. For example, when relative quantifications are employed, an upper bound for any potential ingredient may be set to 1.0, indicating that the target product is to be comprised of only that potential ingredient. Accordingly, the set of potential formulations may be defined via a set of potential ingredients and upper/lower thresholds for each potential ingredient.

In some embodiments, the set of potential formulations may be encodable in a finite vector space, where each potential ingredient in the set of potential ingredients is associated with a separate dimension of the vector space. The formulation encoder 204 may be generally responsible for generating such encodings. The dimensionality of the vector space may be equivalent to the cardinality of the set of potential ingredients. An axis for each dimension of the vector space may be bounded by the upper and lower thresholds of the corresponding potential ingredient. Thus, the finite vector space for a set of potential formulations may be a N-dimensional hyper-rectangle or hyper-box, where Nis a positive integer that corresponds to the cardinality of the set of potential ingredients. This finite vector space may be referred to as the “space of potential formulations,” (or potential formulation space) for the target product. The space may also be referred to as the formulation vector space, or simply the formulation space.

In some embodiments, a distance metric may be imposed on the space of potential formulations that enables measurement of distances and angles in the vector space. That is, a metric may be defined such that inner products and/or distances for pairs of vectors are well defined over the space. In at least one embodiment, the metric may be encoded in a symmetric 2-tensor (e.g., a matrix). In some embodiments, the metric matrix may be a diagonal matrix. The metric matrix may be the identity matrix for the dimensionality of the vector space (e.g., a Euclidean metric). Various embodiments may employ other metrics, e.g., Manhattan, Minkowski, Hamming, and other such distance metrics.

Via the formulation encoder 204, each potential formulation of the set of potential formulations for a target product may be encodable as a vector embedding in the space of potential formulations. Each component of the vector embedding may be associated with a potential ingredient of the set of potential ingredients. The quantification of the potential ingredient may be assigned to the corresponding component in the potential formulation's vector embedding. In some embodiments, the axis for one or more of the dimensions of the space of potential formulations may be discretized via a binning of the axis. In other embodiments, each axis may be a continuous axis of real values. Other vector embeddings of a potential formulation are possible, and the embodiments are not limited to the vector embeddings discussed herein.

Like vector embeddings of a potential formulation, other vector encodings of the set of relevant properties may be employed in the various embodiments. As noted above, the set of relevant properties for the target product may include any of the properties to be introduced, increased, eliminated, or decreased in the formulation (or reformulation) of the target product. That is, the set of relevant properties may include any property to be constrained, controlled for, selected for, selected against, or otherwise targeted for in the target product. Note that the set of relevant properties may not be exhaustive of all the properties of the target product. Rather, the relevant properties of the set of relevant properties may be only those properties that are to be at least partially maximized, minimized, or selected for (or selected against) in the target product's formulation (or reformulation). Values (e.g., target, predicted, and/or empirically observed values) for the set of relevant properties may be encoded by the formulation encoder 206.

The objective function evaluator 208 is enabled to evaluate the objective function upon being provided the target property vector, the property vector encoding the empirically observed values for a particular point the formulation space, and the set of design constraints. The objective function may be undefined for points in the formulation space, or property vectors, that violate one or more design constraints of the set of design constraints. For instance, the objective function evaluator 208 may return an undefined indication when provided with a condition (e.g., a potential formulation or values for the set of relevant properties that would violate one or more design constraints).

In such embodiments, where the cardinality of the set of relevant properties is finite and at least a portion of the relevant properties are quantifiable, values for the set of relevant properties may be encoded be encoded in a data structure or object. The properties encoder 206 may encode a set of relevant properties in a common data structure that is recognized by the other components of the intelligent agent engine 200, e.g., a vector, 1D array, n-tuple, or the like. In one non-limiting example of an encoding employed by the properties encoder 206, each dimension (or component) of the vector (or array or n-tuple) corresponds to a relevant property and the value of the component is set to the quantification of the relevant property. The properties encoder 206 may employ other vector encoding schemas for a set of relevant properties. The properties encoder 206 may transform the set of relevant properties received via the UI manager 202 into the common encoding schema.

The vector encoding schema employed by the properties encoder 206 may define a “property vector space” (or space of property vectors) for the target product and/or the set of relevant properties for the target product. In at least one embodiment, this vector space may be referred to as the property space for the target product. In non-limiting embodiments, each potential property may correspond to a dimension of the property vector space. The axis for each dimension in the property vector space may correspond to the quantification of the property corresponding to the axis. Note that each point in the property vector space may not realizable via a potential formulation. In the above insect repellent example, a portion of the property vector space corresponding to high repellency and low cost may not be achievable via a realizable formulation for the insect repellant.

In some embodiments, a distance metric may be imposed on the property vector space that enables measurement of distances and angles in the vector space. That is, a metric may be defined such that inner products and distances for pairs of vectors are well defined. In at least one embodiment, the metric may be encoded in a symmetric 2-tensor (e.g., a matrix). In some embodiments, the metric matrix may be a diagonal matrix. The metric matrix may be the identity matrix for the dimensionality of the vector space (e.g., a Euclidean metric). Various embodiments may employ other metrics, e.g., Manhattan, Minkowski, Hamming, and other such distance metrics.

The quantification of at least some relevant properties may be predictable without the requirement of manufacturing or preparing at least a prototype instantiation of the product. Other relevant properties may be unpredictable. For such unpredictable relevant properties, at least a prototype instantiation of the product may be manufactured or prepared. Once manufactured or prepared via a potential formulation, the unpredictable property may be measured, observed, or otherwise quantified. That is, the properties of the prototype version of the product may be empirically observed. In the above insect repellent example, the repellency of a potential formulation may be unpredictable, while the cost of the potential formulation is predictable via the potential formulation's set of ingredients. To measure the repellency associated with a potential formulation, a prototype of the insect repellency may be manufactured, and the associated repellency may be measured.

A user may provide the intelligent agent engine 200 the results of any such empirical observations of properties for “experimental” or “prototype” versions of the target product via the UI manager 202. The properties encoder 206 may generate an encoding of such empirically observed properties. In addition to target values for a set of relevant properties, the user may define a set of design constraints for the target product, via the UI manager 202.

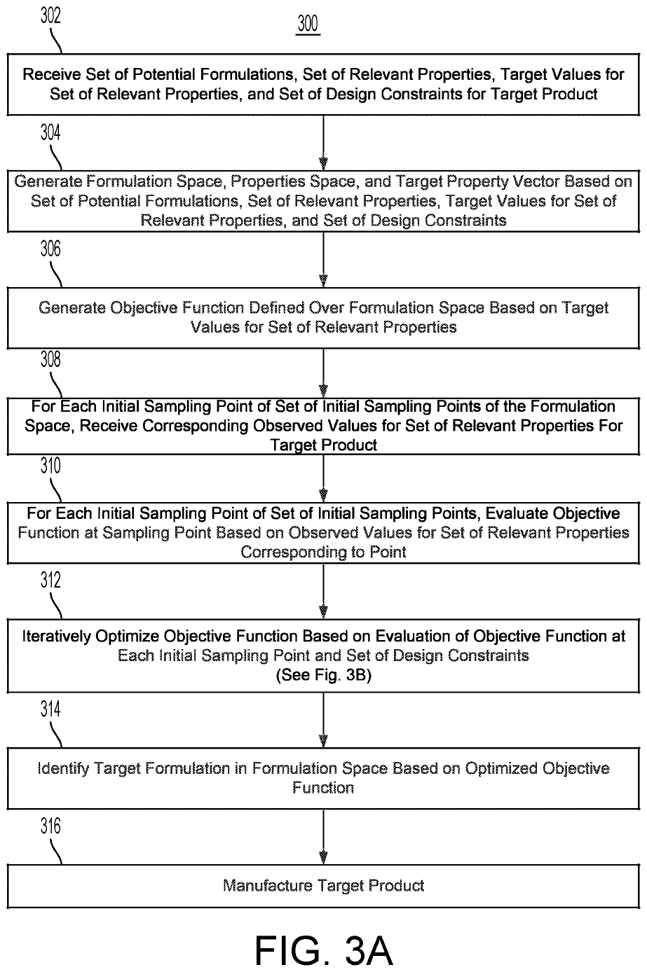

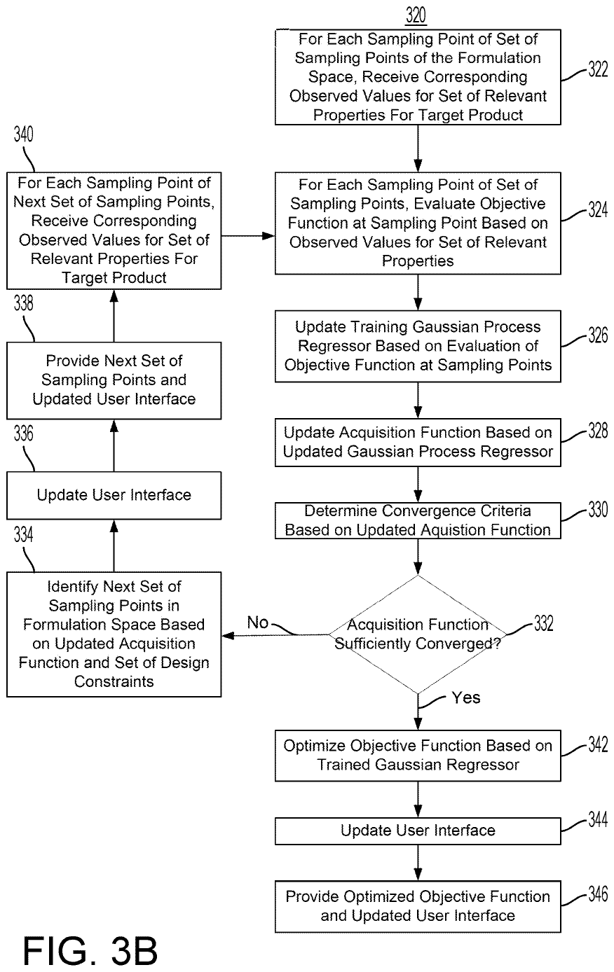

The objective function optimizer 210 may employ various combinations of machine learning and statistical-based techniques that enable the agent to iteratively optimize the objective function. In the non-limiting embodiment of FIG. 2, the objective function optimizer 210 includes a Bayesian optimizer 212 and a Gaussian process regressor 214. As discussed in more detail in conjunction with FIGS. 3A-3B, the Bayesian optimizer 212 may implement a Bayesian optimization process to optimize the objective function. The Bayesian optimizer 212 may employ the Gaussian process regressor 214 to enable the optimization process. The Gaussian process regressor 214 may implement a Gaussian process regression technique to enable the Bayesian optimization process.

### Example Methods

FIG. 3A depicts a flow chart diagram of an example method 300 for providing guidance in the formulation of a target product according to example embodiments of the present disclosure. Although FIG. 3A depicts steps performed in a particular order for purposes of illustration and discussion, the methods of the present disclosure are not limited to the particularly illustrated order or arrangement. The various steps of the method 300 can be omitted, rearranged, combined, and/or adapted in various ways without deviating from the scope of the present disclosure. An intelligent agent implemented by an intelligent agent engine (e.g., intelligent agent engine 140 of FIG. 1 and/or intelligent agent engine 200 of FIG. 2) may perform at least some steps in method 300. The intelligent agent engine may be implemented by a computing system, such as but not limited to system 100 of FIG. 1.

At 302, the intelligent agent can receive a set of potential formulations, a set of relevant properties, target values for the set of relevant properties, and a set of design constrains for a target product. The intelligent agent may receive any of the set of potential formulations, the set of relevant properties, target values for the set of relevant properties, and the set of design constraints through a user employing an interactive user interface (UI). The interactive UI may include a “smart spreadsheet” with an interactive table. The table may be interactive in that a user of a computing device may have access to the table, as well as the intelligent agent. The user may provide information to the intelligent agent by providing values to one or more cells of the table. Likewise, the intelligent agent may provide information to the user by providing value to other cells of the table. The UI may be provided to the user via a UI manager (e.g., UI manager 202 of FIG. 2).

At block 304, a formulation space and a properties space may be generated based on the set of potential formulations, the set of relevant properties, and the set of design constraints. The formulation space and the properties space may each be a vector space. A target property vector may be generated based on the target values for the set of relevant properties. The target property vector may be an embedding vector, in the property space, that encodes the target values for the set of relevant properties. A property encoder (e.g., property encoder 206 of FIG. 2) may generate the target property vector. Each potential formulation of the set of potential formulations may be embedded in the formulation space via a vector encoding of the potential formulation. A formulation encoder (e.g., formulation encoder 204 of FIG. 2) may generate the vector embeddings of the potential formulations.

In at least one embodiment, the formulation space may be updated or modified to “carve out” (or remove) portions of the formulation space that correspond to vector embeddings of potential formulations that would violate one or more of the design constraints of the set of design constraints. The set of relevant properties may be embedded, via a vector encoding, in the properties space. A properties encoder (e.g., properties encoder 206 of FIG. 2) may generate the vector (or scalar embedding for a single relevant property) embedding of the set of relevant properties. Similar to the design constraint-based carve-outs of the formulation space, one or more portions of the properties space may be carved out of the properties space that correspond to vector embeddings of properties that would violate the one or more design constraints.

At block 306, an objective function may be generated based on the values for the set of relevant properties. An objective function evaluator (e.g., objective function evaluator 208 of FIG. 2) may generate the objective function based on a vector (or scalar) encoding of the target values for the set of relevant properties, e.g., the target property vector. The objective function may be a scalar function defined over the formulation space. More particular, the objective function may be a function of a vector (or scalar) embedding of the set of relevant properties corresponding to a potential formulation. Accordingly, the objective function may be a scalar function implicitly defined over the properties space. For example, an evaluation of the objective function may produce more extreme scalar values (e.g., closer to a maximum value or a minimum value of the objective function) when the objective function is evaluated a point corresponding to a property embedding (e.g., scalar or vector) that is more “similar” or “closer” to the vector embedding of the target values for the set of relevant properties, e.g., the target property vector. Such a distance or similarity may be measured via various metrics, e.g., Euclidean distance, cosine similarity, inner product, or the like. That is, the objective function may be a function of a comparison of a vector embedding of the target value of the set of relevant properties and a vector embedding of a set of (empirically observed) relevant properties corresponding to a potential formulation.

At block 308, for each initial sampling point of a set of initial sampling points, values for the set of relevant properties for the target product may be received. The set of sampling points, as well as the values for the sets of relevant properties for the target product, may be provided to the intelligent agent via the UI. The set of initial sampling points may be points (or vector embeddings) in the formulation space. At least some of the properties of each set of relevant properties may have been empirically (or experimentally) measured via manufacturing a prototype product based on the point's corresponding potential formulation (e.g., conducting an experiment). A property encoder (e.g., property encoder 206 of FIG. 2) may encode the received values for sampling point in a vector embedding in the property space.

At block 310, for each initial sampling point of the set of initial sampling points, the objective function may be evaluated at the sampling point based on received values for the set of relevant properties corresponding to the sampling point. The evaluation of the objective function may be an empirical or experimental evaluation of the function based on the empirically observed values for the set of relevant properties. In at least one embodiment, the evaluation of the objective function may be based on a comparison between the vector embedding of the target values for the set of relevant properties and the vector embedding of the corresponding received values for the set of relevant properties. In some embodiments, the comparison may be based on a difference between the two vector embeddings. In at least one embodiment, the comparison may be based on a distance between, an inner product of, or a similarity (e.g., a cosine similarity) of the two vector embeddings. In some embodiments, an objective function evaluator (e.g., objective function evaluator 208 of FIG. 2) may evaluate the objective function. The value for the evaluated function may be provided to the user via the UI. In other embodiments, the user may evaluate the objective function. The user may provide the intelligent agent value for the evaluated function via the UI.

At block 312, the objective function may be iteratively optimized based on the evaluation of the objective function at each initial sampling point of the set of initial sampling points. Various embodiments of iteratively optimizing an objective function are discussed in conjunction with at least method 320 of FIG. 3B. However, briefly here, one or more optimization techniques may be iteratively employed to optimize the objective function. In some embodiments, a Bayesian optimization process may be employed. In at least one embodiment, a Gaussian process regressor may be employed as a sub-process of the Bayesian optimization process.

At block 314, a target formulation for the target product may be selected and/or identified within the formulation space. Selecting the target formulation may be based on the optimized objective function. The target formulation may be provided to the user via the UI. At block 316, the target product may be manufactured and/or prepared based on the selected target formulation.

FIG. 3B depicts a flow chart diagram of an example method 320 for iteratively optimizing an objective function according to example embodiments of the present disclosure. Although FIG. 3B depicts steps performed in a particular order for purposes of illustration and discussion, the methods of the present disclosure are not limited to the particularly illustrated order or arrangement. The various steps of the method 320 can be omitted, rearranged, combined, and/or adapted in various ways without deviating from the scope of the present disclosure. An intelligent agent implemented by an intelligent agent engine (e.g., intelligent agent engine 140 of FIG. 1 and/or intelligent agent engine 200 of FIG. 2) may perform at least some steps in method 320. The intelligent agent engine may be implemented by a computing system, such as but not limited to system 100 of FIG. 1. Method 320 may be a sub-method of method 300 of FIG. 3A. For example, method 320 may be called at block 312 of method 300.

Method 320 may implement a Gaussian optimization process to optimize the objective function. For the purposes of this discussion, the objective function may be referenced as f (x), where x ∈ R^{d}, is a point in the d-dimensional formulation space (R^{d}), and dis a positive integer. Such embodiments are non-limiting, and x could be a variable (or variable type) extending beyond the real (or rational) numbers. For instance, x may encompass a categorical a categorical variable encoding a decision, or it could represent a set of vertices and edges (e.g., a graph data structure) representing a molecular structure. Method 320 enables selecting a target formulation (x_{T}) from the formulation space, such that f(x_{T}) at least approximately optimizes (e.g., maximizes or minimizes) f(x) for all points in the formulation space. In the various embodiments, the objective function may be a black-box function due to at least some of relevant properties being unpredictable properties.

When employing a Gaussian optimization process, method 320 may model the black-box objective function as a random function (e.g., a generalization of a statistical random variable). Method 320 may employ Bayes' theorem, and thus consider a prior distribution of functions (e.g., a distribution of candidate functions that approximate the objective function) for the objective function. As with other applications of Bayes theorem, the prior distribution of functions represents a set of “beliefs” for a “true” representation of the objective function. The objective function may be empirically (or experimentally) evaluated at the sampling points. Via such empirical evaluations and the application of Bayes' theorem on the prior distribution, the set of beliefs is updated via the calculation of a posterior distribution of candidate functions. An acquisition function may be calculated from the posterior distribution. The acquisition function provides a score or utility associated with obtaining an empirical observation of the objective function at that point. The acquisition function may be employed to determine another set of points in the formulation space to sample. Upon evaluating the objective points at this next set of sampling points, the model is updated on these new points yielding a new posterior distribution. This process may iteratively continue until the posterior distribution sufficiently converges.

In at least one embodiment, a Gaussian process regressor is employed to iteratively update the prior/posterior distributions. Briefly, a Gaussian process regressor may include a stochastic technique to estimate a function based on empirical observations of evaluations of the function at the sampling points. In contrast to linear regression techniques, a Gaussian process regressor models each random variable as being distributed via a multivariate normal distribution, where each variable has an independent mean and variance parameter. The means and variance for each random variable are iteratively updated via the iterative empirical evaluations of the objective functions at the iteratively generated sets of sampling points. This iterative estimation of the multivariant normal distribution may be referred to as training the Gaussian process regressor.

At block 322, for each sampling point of a set of sampling points, corresponding values for the set of relevant properties for the target product may be received. The intelligent agent may be provided the values for the set of sampling points via a user employing an interactive user interface (UI). The interactive UI may include a “smart spreadsheet” with an interactive table. The table may be interactive in that a user of a computing device may have access to the table, as well as the intelligent agent. The user may provide information to the intelligent agent by providing values to one or more cells of the table. Likewise, the intelligent agent may provide information to the user by providing value to other cells of the table. The UI may be provided to the user via a UI manager (e.g., UI manager 202 of FIG. 2). Various embodiments for receiving values for the set of sampling points are discussed in conjunction with at least block 308 of method 300.

The set of sampling points may be points (or vector embeddings) in the formulation space. At least some of the properties of each set of relevant properties may have been empirically (or experimentally) measured via manufacturing a prototype product based on the point's corresponding potential formulation. Each received set of relevant properties may be encoded in a vector embedding in the property space. A property encoder (e.g., property encoder 206 of FIG. 2) may generate the vector encoding for each received set of relevant properties. The set of sampling points at block 322 may be a set of initial sampling points (e.g., see block 308 of method 300).

At block 324, for each sampling point of the set of sampling points, the objective function may be evaluated at the sampling point based on the set of relevant properties corresponding to the sampling point. The evaluation of the objective function may be an empirical or experimental evaluation of the function based on the empirically observed set of relevant properties. In at least one embodiment, the evaluation of the objective function may be based on a comparison between the vector embedding of the target values for the set of relevant properties and the vector embedding of the corresponding set of relevant properties. In some embodiments, the comparison may be based on a difference between the two vector embeddings. In at least one embodiment, the comparison may be based on a distance between, an inner product of, or a similarity (e.g., a cosine similarity) of the two vector embeddings. Various embodiments of evaluating an objective function are discussed at least in conjunction with block 310 of method 300.

At block 326, the training of the Gaussian process regressor may be updated based on the (empirical) evaluation of the objective function at the sampling points. In at least one embodiment, a Gaussian processor regressor (e.g., Gaussian process regressor 214 of FIG. 2) may be employed to train the Gaussian process regressor. At block 328, the acquisition function may be updated based on the updated Gaussian process regressor. In various embodiments, a Bayesian optimizer (e.g., Bayesian optimizer 213 of FIG. 2) may be employed to update the acquisition function. In block 330, a convergence criteria may be evaluated based on the updated acquisition function. At decision block 332, it is determined whether to terminate the iterative (and empirical) sampling of the objective function based on the convergence criteria. If the sampling process is to be terminated, method flows to block 242. Otherwise, if the sampling is to be continued, process 320 flows to 334.

At block 334, a next set of sampling points in formulation space is identified (or selected) based on the updated acquisition function and the set of design constraints. In at least one embodiment, if the updated acquisition function identifies sampling points that would violate one or more design constraints of the set of design constraints, the violating points may be removed from the next set of sampling points. The Bayesian optimizer may be employed to determine the next set of sampling points. At block 336, the UI may be updated. For instance, the UI may be updated to indicate the objective function evaluations of block 310 of method 300 and/or block 324 of method 320. The UI may be updated to indicate the identified next set of sampling points. A UI manager (e.g., UI manager 202 of FIG. 2) may be employed to update the UI. At block 338, the next set of sampling points is provided. Providing the next set of sampling point may be performed by providing the updated UI to a user. The UI manager may be employed to provide the next set of sampling points and/or the updated UI. At block 340, for each sampling point in next set of sampling points, observed values for the set of relevant properties may be received. Various embodiments of receiving values for relevant properties are discussed at least in conjunction with block 308 of method 300 and block 322 of method 320. Method 320 may return to block 324 to evaluate the objective function at each of the sampling points in the next set of sampling points.

At block 342, the objective function is optimized based on the trained Gaussian regressor. An objective function optimizer or the Bayesian optimizer may be employed to optimize the objective function. At block 344 the UI may be updated to indicate the objective function. At block 346, the optimized objective function and the updated interface may be provided. At the performance of block 346, method 320 may flow to block 314 of method 300.

### Formulation of Example Target Composite Product

In one non-limiting embodiment, an example target product to be formulated is a mosquito repellant. The target product is a composite product, and the set of ingredients includes three molecules: {vanillin, dimethylcarbate, dimethyl phthalate}. The three potential ingredients may be combined in various ratios. In one embodiment, the vector encoding of a potential formulation may be indicated: {xᵥ, X_{dc}, X_{dp}}, where xᵥ indicates an amount of vanillin in the potential formulation, x_{dc} indicates a, amount of dimethylcarbate in the potential formulation, and x_{dp} indicates an amount of dimethyl phthalate in the potential formulation. The set of basis vectors: {{1,0,0}, {0,1,0}, {0,0,13} may span the formulation space. In one embodiment, the amounts may be relative amounts, such that the boundary condition between vector components: xᵥ+x_{dc}+x_{dp}=1 (or other such relationships) defines “edges” of the formulation space. In other embodiments, the amounts indicated by the vector components are absolute amounts, and the formulation space is unbounded.

The set of relevant properties may include a single relevant property: the repellency of the mosquito repellant. The repellency indicates a time-dependent ability for the repellant to repel mosquitos from a food source. An experimental quantification of the repellency is defined below. A sampling of a point in formulation space may include synthesizing (or manufacturing) a prototype repellant based on a potential formulation with a vector embedding that corresponds to the sampling point. An experiment may be performed to empirically measure the repellency associated with the potential formulation. The experiment may be a controlled experiment, in that the experiment may include two experimental groups of mosquitoes: a control group and a treatment group. Each experimental group may be provided a separate food source. The food source for the treatment group may have been treated with the prototype repellant, while the food source for the control group may have not been treated with the prototype repellant.

Each mosquito in each experimental group is monitored, and in particular, it is noted if a mosquito feeds on its food source, and at what time the mosquito feeds. The feeding (or not feeding) of each mosquito is treated as a random variable subject to a Bernoulli distribution. The cumulative distribution of feeding times for each experimental group is modeled as a sigmoid function (e.g., a logistic function) characterized by two parameters: tₘ is the mean feeding time for the mosquitos in a group and k is the feeding rate. The logistic function is defined as:

$$S ( t , {t}_{m} , k ) ≡ {[ 1 + {e}^{- k ( t - {t}_{m} )} ]}^{- 1} .$$

The feeding times of the mosquitos in each group are measured and the logistic function for each group is determined based on the measured feeding times. A Gaussian process may be employed to regress values for the two parameters for each of the experimental groups. More specifically, for a mosquito in the control group, y_{c}=1 or 0 indicates whether the mosquito feeds or not. The probability for the ith mosquito in the control group to feed may be calculated as:

$${p}_{{C}^{i}} ≡ P [ {y}_{{C}^{i}} = 1 ] := S ( c , 0 , 1 ) ,$$

where c is an unobserved variable that characterizes the probability of feeding. For a mosquito in the treatment group, y_{T}=1 or 0 indicates whether the mosquito feeds or not. The probability for the ith mosquito in the treatment group to feed may be calculated as:

$${p}_{{T}^{i}} ≡ P [ {y}_{{T}^{i}} = 1 ] := S ( t , {t}_{m} , k ) ,$$

where a 2D Gaussian process is used to model the parameters: tₘ and k. The repellency for the potential formulation may be calculated as follows:

$$R ≡ 1 - \frac{{\sum }_{i} {p}_{{T}^{i}}}{{\sum }_{i} {p}_{{C}^{i}}} .$$

Based on the above definition, the maximum value of the repellency is 1.0. Accordingly, the target value for repellency may be set to 1.0. In at least one embodiment, the objective function may be defined, such that the repellency is maximized.

### Additional Disclosure

The technology discussed herein makes reference to servers, databases, software applications, and other computer-based systems, as well as actions taken, and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

Headers have been provided for the convenience of the reader and are not intended to be limiting with respect to the claimed subject matter.

1. A computer-implemented method for manufacturing a target product, the method comprising:
receiving, by a computing system comprising one or more computing devices, an indication of a set of potential formulations for the target product and an indication of target values for a set of relevant properties for the target product;
generating, by the computing system, an objective function over a first domain that is associated with the set of relevant properties, wherein the target values for the set of relevant properties optimizes the objective function over the first domain;
determining, by the computing system, a set of estimating functions for estimating the objective function over a second domain associated with a set of potential functions, wherein determining the set of estimating functions is based on first property data encoding values for the set of relevant properties associated with a first subset of the set of potential formulations;
iteratively updating, by the computing system, the set of estimating functions based on additional property data encoding additional values for the set of relevant properties associated with additional subsets of the potential formulations;
selecting, by the computing system, a target formulation from the set of potential formulations, wherein, based on the updated set of estimating functions, the target formulation substantially optimizes the objective function over the second domain; and
providing, by the computing system, an indication of the target formulation.

2. The method of claim 1, wherein the target product is an insect repellant.

3. The method of claim 1, wherein the target product includes a food item or a beverage.

4. The method of claim 1, wherein the target product includes at least one of a fragrance product, a cosmetic product, or a pharmaceutical product.

5. The method of claim 1, wherein updating the set of estimating functions includes employing a Bayesian optimization process.

6. The method of claim 5, wherein the Bayesian process employs a Gaussian process regressor method.

7. The method of claim 1, wherein values for at least a subset of the set of relevant properties are unpredictable based on a set of ingredients included in a potential formulation.

8. The method of claim 1, wherein the set of relevant properties includes at least one of taste, flavor, smell, color, or texture of the target product.

9. The method of claim 1, wherein the set of relevant properties includes a cost of a set of ingredients included in a potential formulation.

10. The method of claim 1, wherein one or more potential formulations are removed from the set of potential formulations based on a set of design constraints.

11. A computer-implemented method for manufacturing a target product, the method comprising:
receiving, via a user interface (UI) provided by a computing system, first product data for a property of the target product, wherein the first property data corresponds to a first potential formulation of a set of potential formulations for the target product;
selecting a second potential formulation from the set of potential formulations based on processing, at the computing system, a first evaluation of an objective function defined over the set of potential formulations and a target value for the property, wherein the first evaluation of the objective function is based on the first property data and associated with the first potential formulation;
receiving, via the UI, second property for the property of the target product, wherein the second property data corresponds to the second potential formulation;
processing, at the computing system, a second evaluation of an objective function, wherein the second evaluation of the objective function is based on the second property data and is associated with the second potential formulation; and
selecting a target formulation from the set of potential formulations based on processing the second evaluation of the objective function; and
providing, via the UI, the target formulation.

12. A computing system configured to perform the method of claim 1.

13. One or more non-transitory computer-readable media that collectively store instructions that, when executed by a computer system, cause the computer system to perform the method of claim 1.
